(19) United States
(12) Patent Application Publication
(10) Pub. No.: US 2025/0262041 A1
(43) Pub. Date: Aug. 21, 2025

(54) STROKE PREVENTION DEVICES, SYSTEMS, AND METHODS

(71) Applicant: 3DT Holdings, LLC, San Diego, CA (US)

(72) Inventors: Jose A. Navia, Buenos Aires (AR); Ghassan S. Kassab, La Jolla, CA (US); Ane Beatriz Eguzkitza Bazar, Barcelona (ES)

(73) Assignee: 3DT Holdings, LLC, San Diego, CA (US)

(21) Appl. No.: 19/192,243

(22) Filed: Apr. 28, 2025

Related U.S. Application Data
(63) Continuation-in-part of application No. PCT/US23/36060, filed on Oct. 26, 2023.
(60) Provisional application No. 63/419,653, filed on Oct. 26, 2022.

(51) Int. Cl. A61F 2/01; A61F 2/90

(52) U.S. Cl. CPC A61F 2/0103; A61F 2/0105; A61F 2/011; A61F 2/90; A61F 2002/016; A61F 2210/0076; A61F 2250/0039; A61F 2250/006; A61F 2250/0098

(57) ABSTRACT

Deflection devices, systems, and methods for the prevention of stroke configured and negatively charged to filter emboli in the bloodstream and prevent advancement into the artery extending from the aortic arch. Devices may be formed as a single structure or a composite device. Additionally, a retrieval system is provided, including a sleeve catheter and a retrieval device slidably disposed therein, the retrieval device including one or more attachment portions configured to engage at least a portion of a device positioned within an artery extending from the aortic arch.

### PRIORITY

The present patent application is related to, and claims the priority benefit of, International Application No. PCT/US23/36060, filed on Oct. 26, 2023, which is related to, and claims the priority benefit of, U.S. Provisional Patent Application Ser. No. 63/419,653, filed on Oct. 26, 2022, the contents of which are hereby incorporated by reference in their entirety into this disclosure.

### BACKGROUND

A stroke is defined as a rapidly developing loss of brain function due to a disturbance in the blood supply to the brain. This can be due to ischemia (lack of blood supply) caused by thrombosis or embolism or due to a hemorrhage. As a result, the affected area of the brain is unable to function, leading to the inability to move one or more limbs on one side of the body, the inability to understand or formulate speech, or the inability to see one side of the visual field amongst others.

Stroke is ranked as the second leading cause of death worldwide, with an annual mortality rate of about 5.5 million people. For those who survive, it causes chronical disability in up to 50% of them. Stroke affects the elderly the most, resulting in a growing problem given the progressive ageing of the global population, with the proportion of individuals aged ≥85 years being expected to increase threefold worldwide by the year 2035.

Atrial Fibrillation (AF), a heart condition that causes an irregular and often abnormally fast heart rate with a significant reduction in the cardiac output, is known to cause an increase in embolus propensity, and is associated with a six-fold increase in risk of stroke. It represents the most common arrhythmia diagnosed in clinical practice and its prevalence rises steadily from 0.4-1% among the general population to 8% by 80 years of age. Moreover, stroke risk from AF increases exponentially with age, with an estimated 1.45-fold increase for each subsequent decade, reaching an annual risk as high as 23.5% in patients with AF aged 80 to 90 years. Compared with non-AF-related strokes, AF-related strokes are almost twice as likely to be fatal and to cause severe disability in survivors, increasing the length of hospital stay and reducing the likelihood of patients returning to their home, with associated significantly higher mean direct costs per patient.

Ischemic strokes account for 80% of stroke cases while hemorrhagic strokes account for the remaining 20%. Among ischemic strokes, 20% to 30% are cardioembolic. Cardioembolisms may result from three mechanisms: thrombus formation in the left cardiac chamber, release of material from an abnormal valvular surface, or abnormal passage from the venous to the arterial circulation (ie: paradoxical embolism). Echocardiographic and pathologic studies suggest that approximately 90% of strokes with an identified source, can be attributed to thrombus formation in the left atrial appendage.

The mechanism of formation in turn determines the nature and size of cardioembolisms, which are found in a heterogeneous range of types and sizes. The most frequent type of debris in the former corresponds to fibrin and thrombotic material, representing 74%, with tissue-derived material and calcium deposits in lower proportion. Embolisms may have a range of sizes, but those of arising from the cardiac chambers are often large and hence especially likely to cause severe stroke, disability and death. The more common high risk cardioembolic conditions are atrial fibrillation, recent myocardial infarction, mechanical prosthetic valve, dilated myocardiopathy, mitral rheumatic stenosis, and more recently, Transcatheter Aortic Valve Replacement (TAVR).

TAVR has emerged as an alternative, rapidly evolving non-invasive procedure for patients with severe aortic stenosis and medium-to-high surgical risk. By 2025, there will be an estimated 280,000 TAVR procedures performed worldwide. Although this highly promising treatment modality results in less morbidity, shorter time to recovery and similar mortality rates, it is still associated with one of the most devastating and feared complications: cerebral embolism, which in turn may cause stroke. Stroke is associated with a 6-fold increase in mortality in TAVR cohorts, a moderate to severe permanent disability in up to 40% of survivors, a 4.7-fold increased risk of permanent work disability, social isolation and significant financial strain in 80% of stroke survivors, and an increased risk of readmission in patients with stroke after cardiac catheterization.

The time in between the TAVR procedure and the cardioembolic event is an important factor when choosing stroke prevention treatments. Most of them occur in the acute phase following TAVR where cerebral embolic events are frequent. Nonetheless, a significant number of strokes occur between days 2 and 30 post-TAVR, and evidence is mounting on ischemic brain lesions being produced after day 30, with long-term neurological symptoms. Early stroke is mainly due to debris embolization during the procedure, whereas later events are associated with patient specific factors. In a 5-year retrospective cohort study involving 101,430 patients receiving TAVRs in the US, the median time to stroke events was 2.0 days (IQR, 1.0-5.0) days post-TAVR. Of all patients with 30-day stroke events, 1119 patients (48.9%) had a stroke within 1 day and 1567 (68.4%) within 3 days. Of all those with strokes, 2096 patients (91.5%) had ischemic strokes and 128 (5.6%) had hemorrhagic strokes. In the ADVANCE trial, within the first months after TAVR using the CoreValve™, half of the reported strokes occurred on the day of the procedure or the first post-procedural day, and the other half between day 2 and day 30.

Regarding the size of debris, a wide range was identified during TAVR in 86% of patients. The median size of debris was 1 mm (IQR: 0.6 to 1.5 mm) and varied between 0.1 and 9 mm. Fibrin and thrombotic material (size varied between 0.2 and 6.2 mm) was found in 74% of patients. More recently, it has been suggested that microembolisms released after the TAVR periprocedural period may be causing Silent Brain Infarcts (SBIs), finding new ischemic brain lesions in TAVR patients in 74% to 100% of patients on diffusion-weighted magnetic resonance imaging (DW-MRI).

SBI is increasingly recognized in patients with cardiac conditions, particularly AF in elderly patients and those undergoing Transcatheter Aortic Valve Implantation (TAVI). While these infarcts often go unnoticed due to a lack of acute symptoms, they are associated with a threefold increase in stroke risk and are considered a precursor to ischemic stroke. Moreover, accumulating evidence suggests that SBI may contribute to the development of dementia, depression, and cognitive decline, particularly in the elderly population. The burden of SBI is substantial, with studies showing that up to 11 million Americans may experience a silent stroke annually. In AF patients, SBIs are common and can lead to progressive brain damage, even in those receiving anticoagulation therapy. Even though microembolisms may not produce a large proportion of symptomatic strokes, the situation is especially worrying given that TAVRs are being implanted in increasingly younger and lower risk patients, hence potentially increasing the prevalence of dementia.

Emerging data from clinical trials and real-world registries demonstrate the benefits of anticoagulation compared to no therapy, with a clinical advantage of Non-vitamin K Antagonist OACs (NOAC) compared to conventional Anticoagulation with Vitamin K Antagonists (VKAs). Even though these benefits are also observed in elderly patients, non-adherence to Oral Anticoagulant (OAC) treatment, associated comorbidities and additional risk factors can significantly increase the incidence and severity of cerebrovascular accidents. In this age group, a delicate balance may exist between multiple conditions, being thrombotic disease, Chronic Kidney Disease (CKD), cancer, Coronary Artery Disease (CAD) and Heart Failure (HF) some of the most challenging scenarios encountered in clinical practice.

Considering the difficulties with anticoagulant treatments, it is only reasonable to devise Cerebral Embolic Protection Devices (CEPDs) capable of matching or surpassing current OAC efficacy. For strokes triggered by AF or other conditions extended in time, there are no currently-approved CEPDs in the market. Therefore, any long-term device capable of blocking embolisms would be a radical improvement for a large population at risk.

For TAVR-associated strokes, current CEPDs provide protection only during the procedure and up to 2 days after the procedure. Nonetheless, as explained above, risk of stroke may not be limited to the procedure itself or the perioperative period. However, the clogging of current long-term CEPDs impedes them from being used to prevent ischemic strokes >2 days post-TAVR. Therefore, a CEPD able to filter small debris in the long term post-TAVR is needed.

Despite advancements in TAVI technology, cerebrovascular events, including silent brain lesions, continue to pose significant challenges, underscoring the need for improved preventive strategies and therapeutic approaches.

### BRIEF SUMMARY

Aspects of the current subject matter relate to stroke prevention devices, systems and methods directed toward solving some of the above-identified problems, such as based on filtering the highest number of cardioembolism microparticles (10-600 microns) that cause asymptomatic multiple small brain infarcts (SBI).

In accordance with some embodiments described herein, there is provided a device for the prevention of stroke, the device comprising a stent portion and a filter portion. The stent portion has a first end and a second end and is sized and shaped to fit within an artery extending from an aortic arch. The filter portion is positioned at the second end of the stent portion and is sized and shaped to prevent the device from advancing into the artery extending from the aortic arch in which the stent portion may be positioned. The filter portion comprises at least two sets of two or more parallel, convex struts. The at least two sets of two or more parallel convex struts of the device are positioned across an opening defined within the second end of the stent portion and configured to divert an embolus from entering the artery when the first end of the stent portion is positioned within the artery. In at least one exemplary embodiment, when the device is positioned within the artery extending from an aortic arch, the two or more parallel convex struts of a first of the at least two sets of parallel convex struts are positioned either approximately perpendicular to, in a direction of (i.e., approximately parallel with), or in an oblique manner relative to, blood flow within the aortic arch, and the two or more parallel convex struts of a second of the at least two sets of parallel convex struts are positioned perpendicular to the first set of parallel convex struts. In accordance with some embodiments, the filter portion is autoexpandable from a collapsed configuration to an expanded configuration.

In accordance with some embodiments described herein, there is provided a device for the prevention of stroke, the device comprising a filter portion, the filter portion comprising two or more parallel, convex struts positioned either approximately perpendicular to, in a direction of (i.e., approximately parallel with), or in an oblique manner relative to, blood flow within the aortic arch, and at least one flange extending from the filter portion configured to be bendable such that it can be folded up or down from the filter portion.

In accordance with some embodiments described herein, there is provided a device for the prevention of stroke, the device comprising a stent portion, the stent portion comprising a substantially cylindrical shape. In accordance with some embodiments, the stent portion comprises an extension mesh comprising multiple wires. In accordance with some embodiments, the stent portion has a length between about 0.8 cm to about 2.5 cm. In accordance with some embodiments, the stent portion has a diameter between about 6 mm to about 8 mm when the stent portion is in an expanded configuration. In accordance with some embodiments, the stent portion has a diameter between about 1.8 mm to about 2.0 mm when the stent portion is in a compressed configuration.

In accordance with some embodiments described herein, there is provided a device for the prevention of stroke, wherein the device is comprised of a material selected from the group consisting of stainless steel, cobalt-chromium-nickel-molybdenum-iron alloy, tantalum, nitinol, nickel-titanium, polymer materials, and a shape-memory polymer.

In accordance with some embodiments described herein, there is provided a device for the prevention of stroke, the device comprising a filter portion having one or more radiopaque markers positioned thereupon. In accordance with some embodiments, the one or more radiopaque markers are positioned relative to the two or more parallel convex struts. In accordance with some embodiments, when the first end of the stent portion is positioned within the artery extending from an aortic arch, the one or more radiopaque markers facilitate alignment of the device so that the two or more parallel convex struts are positioned either approximately perpendicular to, or in a direction of (i.e., approximately parallel with), or in an oblique manner relative to, blood flow within the aortic arch. In at least one exemplary embodiment of a device for the prevention of stroke of the present disclosure, the diameter of each of the two or more parallel convex struts is between about 0.25 mm and about 1.0 mm, inclusive. In another embodiment, the two or more parallel convex struts are positioned between about 0.75 mm to about 1.0 mm, inclusive, from one another. In yet another embodiment, the two or more parallel convex struts are flexible.

In accordance with some embodiments described herein, there is provided a device for the prevention of stroke, the device comprising a filter portion, the filter portion comprising a first set of two or more parallel, convex struts positioned either approximately perpendicular to, in a direction of (i.e., approximately parallel with), or in an oblique manner relative to, blood flow within the aortic arch. The two or more parallel, convex struts are negatively charged, for example by coating them with different minerals, nanoparticles, and biological components (e.g., negative nanoparticles of graphene OX), and in some embodiments, in order to repel the clots that are also negatively charged, as well as reduce the maximum possible hemolysis.

In accordance with some embodiments described herein, there is provided a device for the prevention of stroke, wherein the device is coated with graphene oxide, plus bovine serum albumin and/or gold magnetic nanoparticles and/or silver magnetic nanoparticles, with a negative charge within physiological limits.

In accordance with some embodiments described herein, there is provided a device for the prevention of stroke, the device comprising a stent portion and a filter portion. The stent portion has a first end and a second end and is sized and shaped to fit within an artery extending from an aortic arch. The filter portion is removably attached to the second end of the stent portion and is sized and shaped to prevent the device from advancing into the artery extending from the aortic arch in which the stent portion may be positioned. The filter portion comprises two or more parallel, convex struts and is configured such that when the filter portion is attached to the second end of the stent portion, the two or more parallel convex struts of the device are positioned across an opening defined within the second end of the stent portion and configured to divert an embolus from entering the artery when the first end of the stent portion is positioned within the artery. In at least one exemplary embodiment, when the device is positioned within the artery extending from an aortic arch, the two or more parallel convex struts are positioned either approximately perpendicular to, in a direction of (i.e., approximately parallel with), or in an oblique manner relative to, blood flow within the aortic arch. Once the device has been positioned within the artery extending from an aortic arch, the filter portion can be removed from the stent portion and from the patient leaving only the stent portion remaining in the artery.

In accordance with some embodiments described herein, there is provided a retrieval system for the prevention of stroke of the present disclosure, the system comprising at least one device for the prevention of stroke, a sleeve catheter and a retrieval device. The at least one device comprises a stent portion having a first end and a second end (the stent portion sized and shaped to fit within an artery extending from an aortic arch), a filter portion positioned at the second end of the stent portion (the filter portion sized and shaped to prevent the device from advancing into the artery extending from the aortic arch in which the first end of the stent portion may be positioned), and two or more parallel convex struts positioned across an opening defined within the second end of the stent portion, the two or more parallel convex struts configured to divert an embolus from entering the artery when the first end of the stent portion is positioned within the artery. The sleeve catheter is configured for intravascular insertion and advancement, the sleeve catheter comprising a proximal end, an open distal end, and a lumen extending therebetween, and the retrieval device slidably disposed within the lumen of the sleeve catheter, the retrieval device comprising a proximal end for manipulation by a user and a distal end comprising one or more second attachment portions, wherein each of the one or more second attachment portions of the retrieval device are configured to engage the first attachment portion of the filter portion of the device. In accordance with some embodiments, the system comprises a conical dilator sized and shaped to slidingly engage the hypotube. In accordance with some embodiments, the conical dilator comprises a tapered distal and a proximal end. In accordance with some embodiments, the folder has an inner diameter, and wherein the tapered distal end of the conical dilator is sized and shaped to fit within the inner diameter of the folder. In accordance with some embodiments, when the device is positioned within the artery extending from an aortic arch, the two or more parallel convex struts either approximately perpendicular to, in a direction of (i.e., approximately parallel with), or in an oblique manner relative to, blood flow within the aortic arch. In accordance with some embodiments, the retrieval device of the system comprises one or more wires. In accordance with some embodiments, the system comprises two devices for prevention of a stroke. In accordance with some embodiments, the first attachment portion of the filter portion comprises a screw tip and a first magnet and the second attachment portion of the retrieval device comprises a screw hole and a second magnet, and the screw tip and the first magnet of the first attachment portion are configured to securely engage with the screw hole and the second magnet of the second attachment portion, respectively. In accordance with some embodiments, the second attachment portion of the retrieval device comprises a lace component and the first attachment portion of the filter portion comprises a hook tip configured to engage the lace component of the retrieval device.

In accordance with some embodiments described herein, there is provided a method for preventing stroke of the present disclosure, the method comprising the steps of introducing a device for preventing stroke into a body, navigating the device within the body until the device reaches an aortic arch, and positioning the device within a first vessel branching from the aortic arch so that the two or more convex struts are positioned either approximately perpendicular to, or in a direction of (i.e. approximately parallel with), or in an oblique manner relative to, blood flow within the aortic arch. In another embodiment, in the step of introducing a device for preventing stroke into a body, the device comprises an stent portion having a first end and a second end, a filter portion coupled with the second end of the stent portion and sized and shaped to prevent the device from advancing into the artery extending from the aortic arch in which the first end of the stent portion may be positioned, and two or more convex struts positioned across an opening defined within the second end of the stent portion. Here, the stent portion may be sized and shaped to fit within an artery extending from the aortic arch and/or the two or more convex struts of the device may be configured to divert an embolus from entering the artery when the first end of the stent portion is positioned within the artery. In accordance with some embodiments, the step of positioning the device is performed by aligning the device within the vessel by detecting one or more radiopaque markers positioned upon the device. Furthermore, placement of the device within the first vessel does not significantly affect upstream blood flow patterns. In an additional embodiment, the step of positioning the device comprises positioning the device within an innominate artery.

In accordance with some embodiments described herein, there is provided a method for preventing stroke of the present disclosure, the method comprising the steps of introducing a second device for preventing stroke into the body; navigating the second device within the body until the second device reaches the aortic arch; and positioning the second device within a second vessel branching from the aortic arch. In this manner, two or more convex struts of the second stent are positioned either approximately perpendicular to, in a direction of (i.e., approximately parallel with), or in an oblique manner relative to, blood flow within the aortic arch. In another embodiment, the step of positioning the second device comprises positioning the second device within a left common carotid artery. In accordance with some embodiments, the step of positioning the first device comprises positioning the first device within an innominate artery (or right brachiocephalic trunk), wherein the first device is capable of diverting an embolus from entering the innominate artery and thus the right common carotid artery and the right subclavian artery, and the second device is capable of diverting the embolus from entering the left common carotid artery. Additionally, a third device may be positioned within a left subclavian artery, which gives rise to the left vertebral artery (LVA). In general, the LVA is more dominant than the right vertebral artery (RVA) and therefore has a larger posterior brain vascular territory.

In accordance with some embodiments described herein, there is provided a method for preventing stroke of the present disclosure, the method comprising the step of anchoring the device within the first vessel by deploying the stent portion and the filter portion of the device. Additionally, the step of anchoring the device within the first vessel may further comprise moving the stent portion from a collapsed position to an expanded position and moving the filter portion from a collapsed position to an expanded position. In accordance with some embodiments, the method further comprises the steps of retrieving the filter portion from the first vessel and removing the filter portion from the body. In accordance with some embodiments, the steps of retrieving the filter portion from within the first vessel and removing the filter portion from the body further comprise the steps of: introducing a retrieval system into the body, navigating the sleeve catheter within the body until the open distal end of the sleeve catheter reaches an aortic arch, advancing the distal end of the retrieval catheter through the open distal end of the sleeve catheter so that the one or more attachment portions engage the filter portion of the device, rotating the filter portion to disengage it from the stent portion, and withdrawing the filter portion from the first vessel, and withdrawing the filter portion and the retrieval system from the body. In accordance with some embodiments, the step of introducing a retrieval system into the body further comprises the retrieval system comprising a sleeve catheter configured for intravascular insertion and advancement, the sleeve catheter comprising a proximal end, an open distal end, and a lumen extending therebetween, and a retrieval device slidably disposed within the lumen of the sleeve catheter, the retrieval device comprising a proximal end for manipulation by a user and a distal end comprising one or more attachment portions, each of which are configured to engage the filter portion of the device.

### BRIEF DESCRIPTION OF THE DRAWINGS

FIG. 1 shows a diagram of at least a portion of an aorta, according to the present disclosure;

FIGS. 2A-2E show an exemplary embodiment of a device for the prevention of stroke, according to the present disclosure;

FIGS. 2F-2G show an exemplary embodiment of a device for the prevention of stroke, according to the present disclosure;

FIGS. 3A-3C show an exemplary embodiment of a device comprising at least one foldable flange, according to the present disclosure;

FIGS. 4A-4B show an exemplary embodiment of a composite device for the prevention of stroke, according to the present disclosure;

FIG. 4C shows an exemplary embodiment of the attachment mechanisms of the device of FIGS. 4A and 4B;

FIGS. 5A-5B show an exemplary embodiment of a device for the prevention of stroke, according to the present disclosure;

FIGS. 5C-5D show an exemplary embodiment of the attachment mechanisms of the device of FIGS. 5A and 5B;

FIG. 6A shows exemplary devices for the prevention of stroke positioned within arteries extending from a portion of an aorta with the convex struts in alignment with blood flow, according to the present disclosure;

FIG. 6B shows exemplary devices for the prevention of stroke positioned within arteries extending from a portion of an aorta with the convex struts in alignment approximately perpendicular to blood flow, according to the present disclosure;

FIG. 6C shows a blown up, semi-cut-away view of a portion of an exemplary device for the prevention of stroke positioned within arteries;

FIG. 7 shows an exemplary embodiment of a device for the prevention of stroke, according to the present disclosure;

FIGS. 8A and 8B show an exemplary system of the present disclosure with portions thereof being moved to allow for device deployment, according to the present disclosure;

FIGS. 9A and 9B show at least a portion of an exemplary system for preventing stroke, said system comprising a conical dilator useful to facilitate removal of at least a portion of the exemplary system from the body, according to the present disclosure;

FIGS. 9C and 9D show additional embodiments of an exemplary system for preventing stroke, according to the present disclosure;

FIGS. 10A-10E show various steps of a method for positioning a device within a body, according to the present disclosure;

FIG. 11 shows at least a portion of an exemplary system for retrieving a portion of a device previously positioned within a body, according to the present disclosure;

FIGS. 12A and 12B show various steps of a method for retrieving a portion of a device previously positioned within a body, according to the present disclosure;

FIGS. 13A and 13B show embodiments of an attachment portion of an exemplary system for retrieving a device previously positioned within a body; and

FIG. 14 shows a flow chart of an exemplary method for preventing stroke according to the present disclosure.

An overview of the features, functions and/or configurations of the components depicted in the various figures will now be presented. It should be appreciated that not all of the features of the components of the figures are necessarily described. Some of these non-discussed features, such as various couplers, etc., as well as other discussed features, are inherent from the figures themselves. Other non-discussed features may be inherent in component geometry and/or configuration.

### DETAILED DESCRIPTION

For purposes of promoting an understanding of the principles of the present disclosure, reference will now be made to the embodiments illustrated in the drawings, and specific language will be used to describe the same. It will nevertheless be understood that no limitation of the scope of this disclosure is thereby intended. Furthermore, numerous specific details are set forth in order to provide a thorough understanding of the present disclosure. Particular examples may be implemented without some or all of these specific details. In other instances, well known devices or processes have not been described in detail so as to not unnecessarily obscure the present disclosure. It will thus be appreciated that aspects and features of apparatus and methods discussed herein which are not described in detail may be implemented in accordance with any conventional techniques for implementing such aspects and features.

Various systems, methods and techniques of the present disclosure will sometimes describe a connection between two components. Words such as attached, affixed, coupled, connected, and similar terms with their inflectional morphemes are used interchangeably, unless the difference is noted or made otherwise clear from the context. These words and expressions do not necessarily signify direct connections but include connections through mediate components and devices. It should be noted that a connection between two components does not necessarily mean a direct, unimpeded connection, as a variety of other components may reside between the two components of note. Consequently, a connection does not necessarily mean a direct, unimpeded connection unless otherwise noted. Furthermore, wherever feasible and convenient, like reference numerals are used in the figures and the description to refer to the same or like parts or steps. Additionally, the drawings are in a simplified form and not to precise scale.

The disclosure of the present application provides various devices, systems, and methods for the prevention of stroke. The devices, systems, and methods disclosed herein facilitate stroke prevention, in part, by addressing specific areas of the heart and diverting the trajectories of blood clots away therefrom with minimal to no influence on resistance of blood flow through such areas and/or significantly affect upstream blood flow patterns.

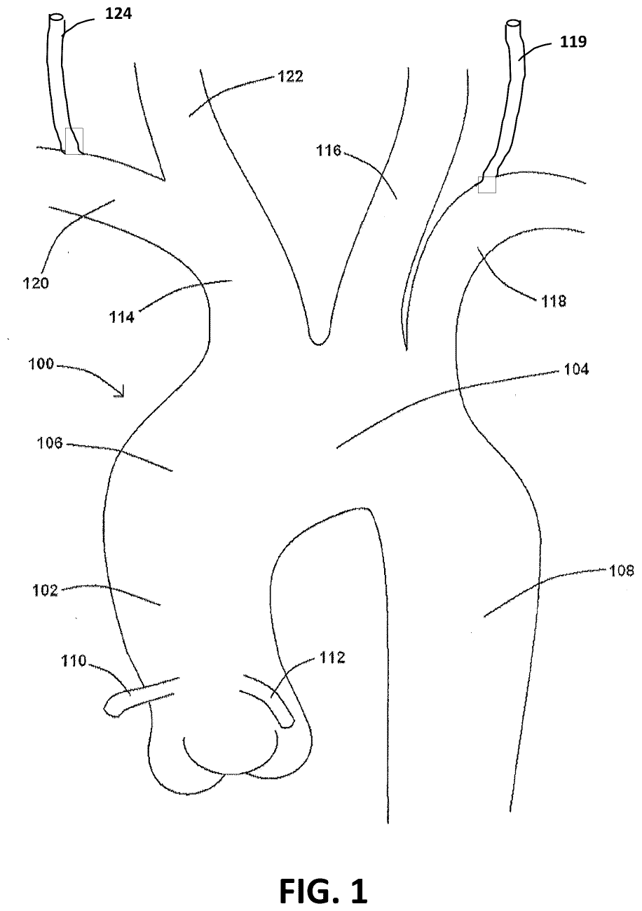

A diagram of at least a portion of an exemplary aorta is shown in FIG. 1. An aorta 100 is the main trunk of a vascular system which conveys oxygenated blood to the tissues of a body. It begins at the upper part of the left ventricle, where it may be approximately 3 cm in diameter in an adult human. As shown in FIG. 1, and at the union of the ascending aorta 102 with the aortic arch 104 (or the “arch of aorta”), the caliber of the vessel is increased, owing to a bulging of its right wall. This dilatation is termed the aortic bulb 106 (or bulb of the aorta), and on transverse section shows a somewhat oval figure. The ascending aorta 102 is contained within the pericardium and is enclosed in a tube of the serous pericardium. It ascends for a short distance (the ascending aorta 102 is about 5 cm in length in an adult human), arches backward, and then descends within the thorax and abdomen (the descending aorta 108) and ends into the right and left common iliac arteries (about 1.7 cm in diameter in an adult human). The right coronary 110 and the left coronary 112, as shown in FIG. 1, branch from the ascending aorta 102.

There are three arteries that branch from the aortic arch 104, namely the innominate artery (or right brachiocephalic trunk) 114, the left common carotid artery 116, and the left subclavian artery 118. As shown in FIG. 1, the left vertebral artery (LVA) 119 branches off from the left subclavian artery 118. The LVA 119 irrigates the greater posterior part of the brain. Instead of arising from the highest part of the aortic arch 104, these branches may spring from the commencement of the aortic arch 104 or the upper part of the ascending aorta 102. The distance between the aortic arch 104 or the upper part of the ascending aorta 102 at their origins may be increased or diminished, the most frequent variation being the approximation of the left common carotid artery 116 toward the innominate artery 114. In addition, and as shown in FIG. 1, the innominate artery 114 branches into the right subclavian artery 120 and the right common carotid artery 122, and the right vertebral artery (RVA) 124 branches off from the right subclavian artery 120.

Ischemic strokes, the most common type of stroke, occur when blood clots or other debris are swept through the bloodstream and lodge in one or more of the aortic branches 114, 116, 118. As the innominate and left common carotid arteries 114, 116, 118, ultimately supply blood to the brain, the partial or complete blockage thereof reduces or inhibits blood flow to the brain, thus increasing the risk of ischemic stroke. Ejection dynamics of blood clots from the left ventricle is diverse and random, with clots having different release velocities at different stages of the cardiac cycle. Furthermore, blood clots can vary in size—typically in the range of about 2 mm to about 6 mm—which can also have a significant effect on clot velocity and their flow patterns as they leave the heart. In addition, the hemodynamics in the aortic arch 104 are typically characterized as complex flow patterns due to the arch curvature and branches 114, 116, 118. Accordingly, clot trajectory is a complex function of aortic flow conditions, discrete phase behavior of clots, and their dynamic interactions. To prevent ischemic stroke, not only must clots be prevented from lodging within the aortic branches 114, 116, 118, but the solution must be mindful of the complexity of the aortic flow field and not generate a substantial resistance to flow therethrough.

The devices, systems, and methods of the present application are configured to maintain a balance between efficacy in deflecting blood clots from an artery extending from the aortic arch 104 and affecting minimal influence on resistance to blood flow therethrough. In this manner, such deflection devices, systems and methods can ensure diversion of blood clots away from the aortic branches 114, 116, 118, rather than blocking clots on the device and thereby obstructing the underlying arteries.

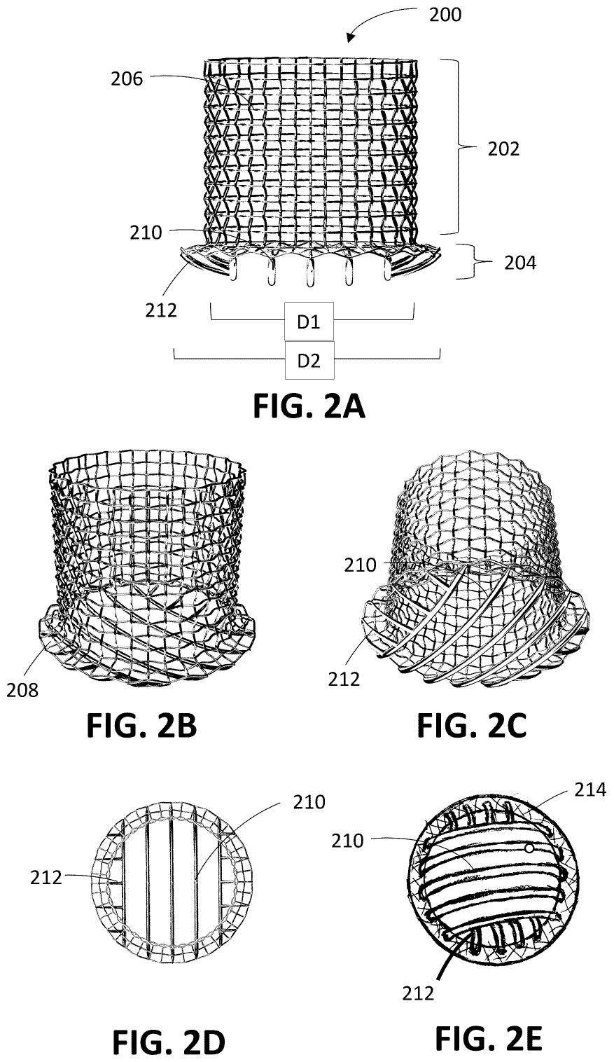

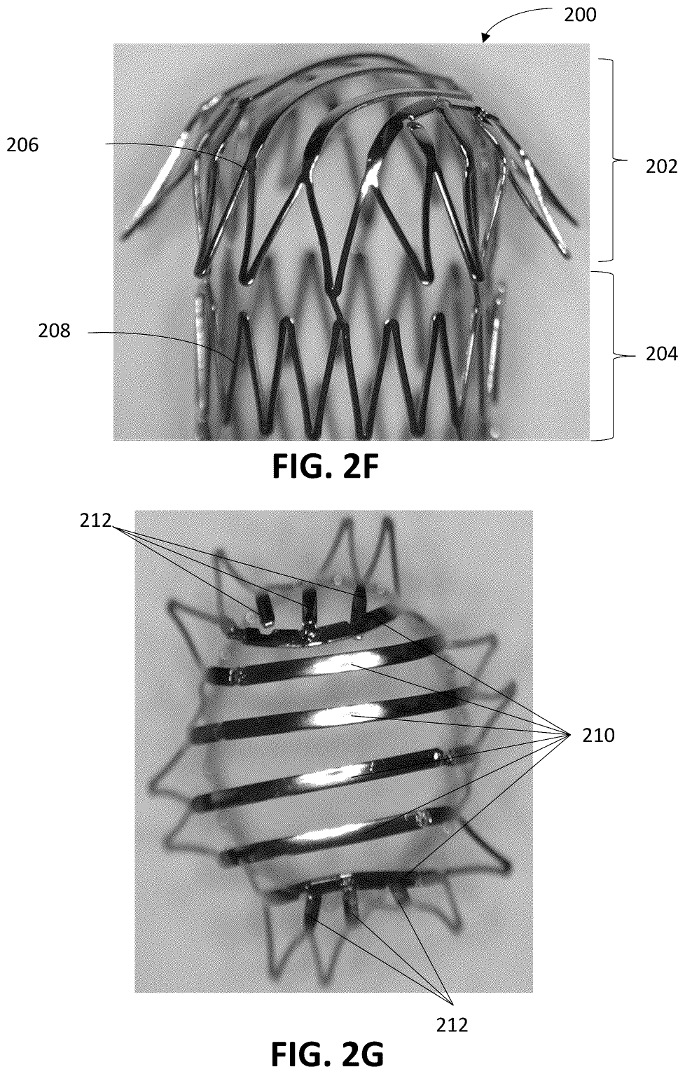

FIGS. 2A-2E show an exemplary embodiment of a device of the present application for the prevention of stroke. In application, such device (and any embodiments thereof) may be used with one or more of the aortic branches 114, 116, 118 to deflect the trajectory of blood clots destined for the structures of the aorta 100 with negligible change in blood flow resistance. As shown in FIG. 2A, an exemplary device 200 may comprise a stent comprising a stent portion 202 and a filter portion 204. Stent portion 202, as shown in FIG. 2A, may comprise a cylindrical stent sized and shaped to fit securely within an aortic branch. An exemplary stent portion 202 may comprise, for example, extension mesh 206 comprising multiple wires as shown in FIG. 2A. Filter portion 204 may comprise an inner diameter (shown as D1 in FIG. 2A) and an outer diameter (shown as D2), whereby D2 is larger than D1. In at least one embodiment, device 200 is collapsible, similar to a traditional stent. Alternatively, or additionally, the device 200 (or independent components thereof) may be autoexpandable to facilitate secure anchoring within an artery and/or the long-term stability of the device 200 after placement.

In at least one embodiment of device 200 of the disclosure of the present application, device 200 comprises an autoexpandable metallic stent comprising a proximal flange (filter portion 204) and a distal cylindrical tube (stent portion 202). In an exemplary embodiment, stent portion 202 is approximately 0.8 cm to 2.5 cm in length. In at least one embodiment of device 200, the diameter of the stent is approximately 6 to 8 mm. Suitable material for a device 200 includes but is not limited to, stainless steel, cobalt-chromium-nickel-molybdenum-iron alloy, tantalum, nitinol, nickel-titanium, polymer materials, and various shape-memory polymers known in the art, including polyurethane, polytetrafluoroethylene or polytetrafluoroethene (PTFE), or another synthetic material.

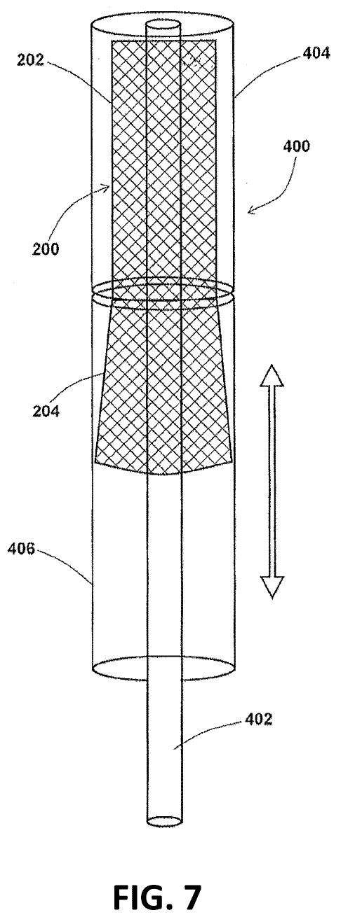

Filter portion 204, as shown in the exemplary embodiments shown in FIGS. 2A-2C, comprises filter mesh 208 comprising multiple wires. The filter portion 204 may comprise any length and/or diameter that is effective to impede the progression of the device 200 within an artery when positioned within a body. In at least one embodiment, the filter portion 204 is between about 2 mm and about 5 mm in length. Furthermore, the filter portion 204 may be configured to move between a collapsed position having a smaller diameter for delivery and/or retrieval of the device 200 (see FIG. 7) and an expanded position having a larger diameter (see FIG. 2A). For example, in at least one embodiment, the filter portion 204 is comprised of an autoexpandable material such that when the filter portion 204 is released from a delivery mechanism, it automatically moves into the expanded position to assist in anchoring the device 200 within an artery of interest.

Alternatively, as shown in FIGS. 2F-2G, device 200 may be configured such that the extension mesh 206 of stent portion 202 and filter mesh 208 of the filter portion 204 are configured in a zig-zag pattern.

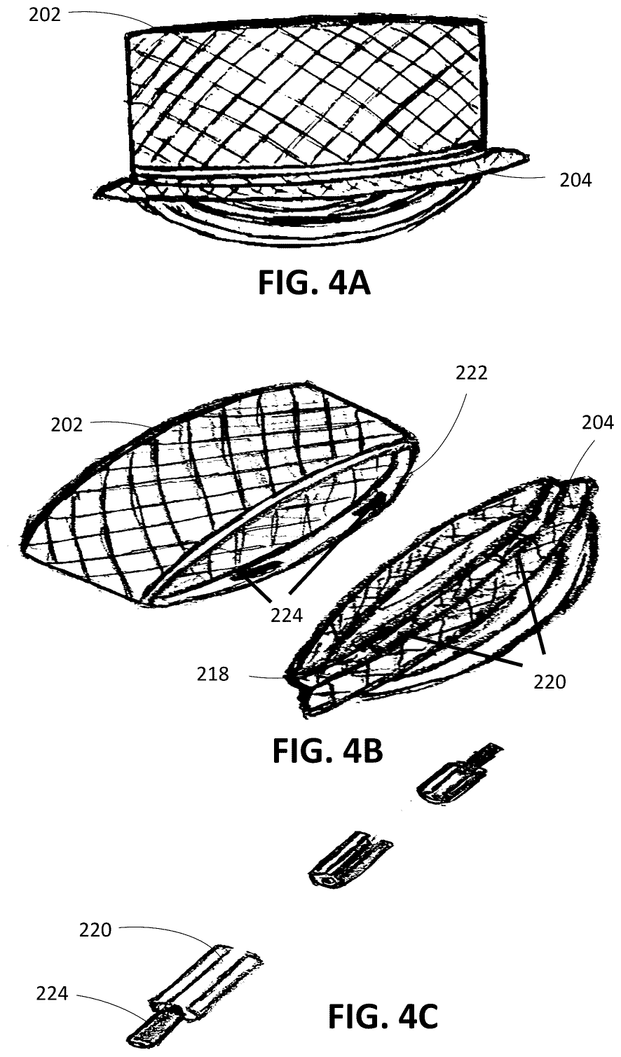

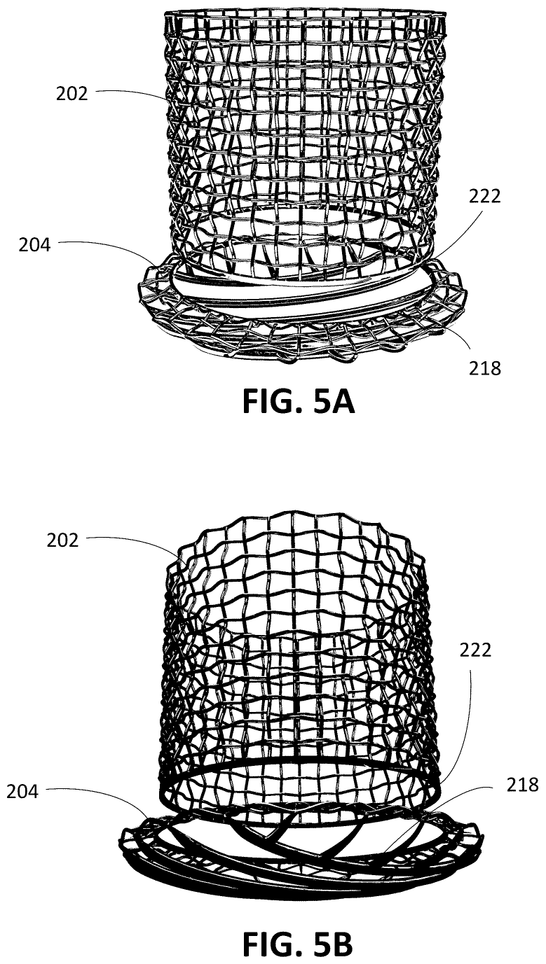

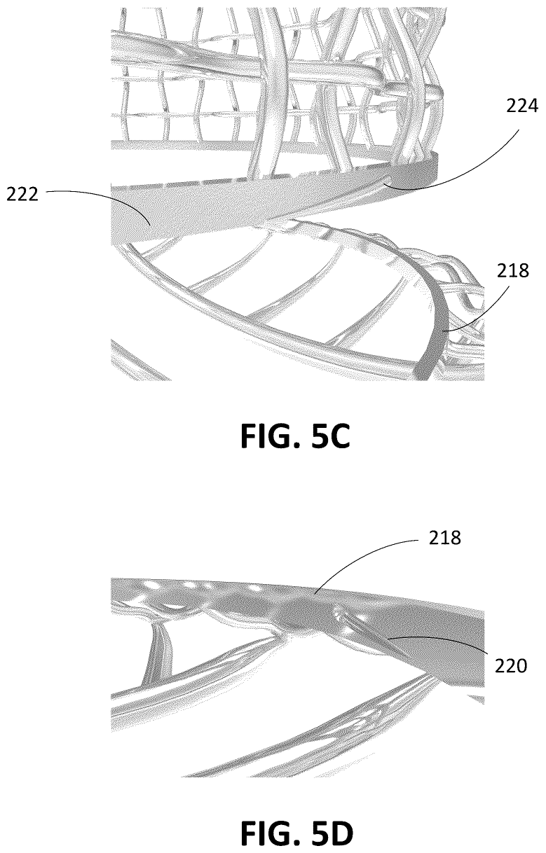

In an exemplary embodiment of device 200 of the disclosure of the present application, the device 200 may be formed as a single structure as shown in FIGS. 2A-2E (or 2F-2G). Alternatively, in another exemplary embodiment of device 200 of the disclosure of the present application, device 200 may be a composite device in which stent portion 202 and filter portion 204 are separate components releasably connected to one another, as shown in FIGS. 4A-4C and 5A-5D. Stent section 202 and filter section 204 may be releasably connected to one another by any appropriate means. For example, as depicted in FIGS. 4A-5D, filter section 204 may include a rim 218 having a groove or channel 220 provided on an inner surface of rim 218 rim, and stent section 202 may include a rim 222 having at least one protrusion 224 provided on an outer surface of rim 222, wherein the channel 220 and the protrusion 224 are configured to operably engage one another upon placement of the rim 218 of filter section 204 within the rim 222 of stent section 202 and rotation of the filter section, thereby latching the filter section 204 and stent section 202 together. The filter section 204 can then be rotated in an opposite direction to disconnect it from the stent section 202 such that the filter section 204 can be removed from the stent section 202. Furthermore, rim 218 and rim 222 may be configured to move between a collapsed position having a smaller diameter for delivery and/or retrieval of the device 200 and an expanded position having a larger diameter similar to filter section 204 and stent section 202. The channel 220 and at least one protrusion 224 are depicted on the filter section 204 and stent section 202 respectively, but it is contemplated that the arrangement can reversed such that the channel 220 is on the stent portion 202 and the at least one protrusion is on the filter section 204. Alternatively, other fixing methods may be used to connect filter section 204 and stent section 202, such as magnetic coupling, or other similar methods.

As shown in FIGS. 2A-2F, the device 200 also comprises two or more convex struts 210 operable to divert, for example, an embolus, from entering the inner portion of device 200 (the inner portion defined by stent portion 202) while still allowing blood to flow therethrough without significantly affecting flow resistance. Convex struts 210 are one example of such an embolus diversion portion of device 200, noting that other embodiments of an embolus diversion not comprising convex struts 210 may be useful with device 200. For example, and instead of convex struts 210, an exemplary embolus diversion portion may comprise a mesh (similar to, for example, extension mesh 206 and/or flange mesh 208), whereby such a mesh is operable to divert an embolus from entering the inner portion of device 200.

Convex struts 210, in an exemplary embodiment, are positioned along device 200 to cover the proximal orifice of the cylindrical stent (device 200). In at least one embodiment of a device 200 of the disclosure of the present application, the diameter of each convex strut 210 is approximately 0.25 mm to 1.0 mm, and the distance between each convex strut 210 is approximately 0.75 mm to 1.0 mm. In at least one exemplary embodiment, the diameter of each convex strut 210 is approximately 0.75 mm and the distance between each convex strut 210 is approximately 0.75 mm, which has been found to provide beneficial deflection efficacy with respect to emboli while affecting only negligible change in flow resistance through the underlying artery.

The device 200 also comprises two sets of at least two or more lateral struts 212, the first set of two or more lateral struts 212 extending from one of the outermost convex struts 210 in a direction away from the rest of the convex struts 210 to filter mesh 208, and the second set of two or more lateral struts 212 extending from the other of the outermost convex strut 210 in a direction away from the rest of the convex struts 210 to filter mesh 208 as shown in FIGS. 2A-2E. The lateral struts 212 are operable to divert, for example, an embolus, from entering the inner portion of device 200 (the inner portion defined by stent portion 202) while still allowing blood to flow therethrough without significantly affecting flow resistance.

Lateral struts 212 are positioned to cover the gap between the outermost convex struts 210 and filter mesh 208. In at least one embodiment of a device 200 of the disclosure of the present application, the diameter of each lateral strut 212 is approximately 0.25 mm to 1.0 mm, and the distance between each lateral strut 212 is approximately 0.75 mm to 1.0 mm. In at least one exemplary embodiment, the diameter of each lateral strut 212 is approximately 0.75 mm and the distance between each lateral strut 212 is approximately 0.75 mm, which has been found to provide beneficial deflection efficacy with respect to emboli while affecting only negligible change in flow resistance through the underlying artery.

It will be appreciated that the number of convex struts 210 and lateral struts 212 present on the device 200 may be customized according to a user's preferences and/or patient specifications. Furthermore, each convex strut 210 and/or lateral strut 212 of the device 200 need not be configured identically; indeed, device 200 may be configured to employ various combinations of convex strut 210 and/or lateral strut 212 diameters, intervals, and heights. Moreover, the convex struts 210 and/or lateral struts 212 may also comprise varying cross-sectional areas and/or a non-spherical profile of the convex envelope. Convex struts 210 and lateral struts 212 may comprise material the same and/or similar to the material used to prepare other portions of device 200, and may also be a combination of a metal plus polyurethane, polytetrafluoroethylene or polytetrafluoroethene (PTFE), or another synthetic material.

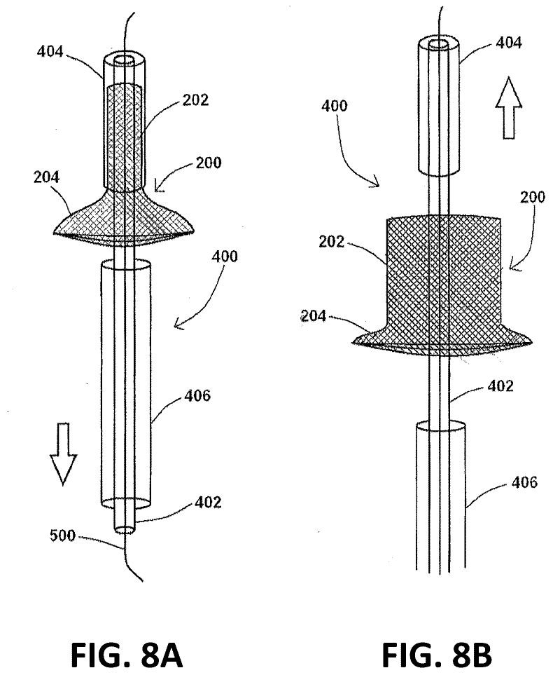

In at least one embodiment, convex struts 210 and lateral struts 212 may be semi-rigid or flexible in order to allow the removal of a hypotube 402 (see FIGS. 7-8B) and/or allow the passage of a catheter stent device, including device 200, for stenting the carotid artery, for example, if it develops an atherosclerotic plaque. In an exemplary embodiment, the shape of the convex struts 210 and/or lateral struts 212 can be convex or semi-convex in order to be easily and constantly “washed” by the aortic blood flow and therefore avoid local thrombosis. If an embolus lands on a strut, the strut shape will also allow it to wash off to the periphery not only preventing the embolus from entering the brain vascular system, but also deflecting the embolus away from the ostium of the artery to ensure the blood flow therethrough does not become restricted or blocked (i.e., the embolus does not stick to the convex struts 210 or lateral struts 212, but rather deflects off).

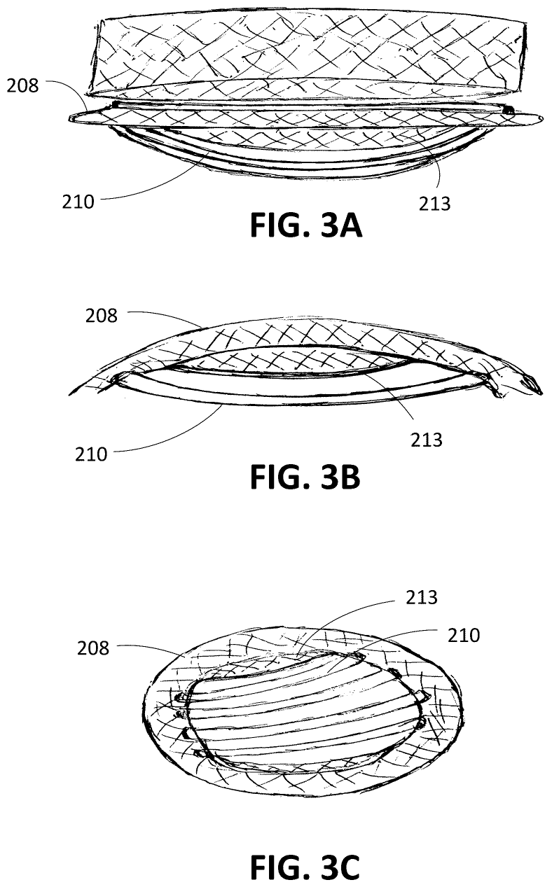

Lateral struts 210 are one example of an embolus diversion portion of device 200 to cover the gap between the outermost convex struts 210 and filter mesh 208, noting that other embodiments of an embolus diversion not comprising lateral struts 212 may be useful with device 200. For example, and instead of lateral struts 212, an exemplary embolus diversion portion may comprise two or more lateral mesh flanges 213 (similar to, for example, extension mesh 206 and/or flange mesh 208), as shown in FIGS. 3A-3C, whereby such lateral mesh flanges 213 extend from the filter mesh 208 positioned proximal the outermost convex struts 210, and are configured to be foldable such that when folded the lateral mesh flanges 213 cover the gap between the outermost convex struts 210 and filter mesh 208, and thus operable to divert, for example, an embolus, from entering the inner portion of device 200 (the inner portion defined by stent portion 202) while still allowing blood to flow therethrough without significantly affecting flow resistance.

In at least one embodiment, some portion or all of the device 200, but at least convex struts 210, lateral struts 212 and/or lateral mesh flanges 213, are negatively charged, for example by coating them with a coating 215 that may comprise different minerals, nanoparticles, and biological components (e.g., negative nanoparticles of graphene OX), in order to repel the clots that are also negatively charged, as well as reduce the maximum possible hemolysis. For example, highly stable and biocompatible supramolecular-aptamer functionalized graphene oxide (GO) nanosheets may be used to coat convex struts 210, lateral struts 212 and/or lateral mesh flanges 213. Supra-TBA_{15/29}-GO has good biocompatibility and low cytotoxicity toward mammalian cells. Other coating materials could include Chitosan, Bovine serum protein, etc., all of which decrease hemolysis thrombosis and cytotoxicity, and are negatively charged. In at least one exemplary embodiment, some portions or all portions of the device 200, but at least convex struts 210, lateral struts 212 and/or lateral mesh flanges 213, are covered with a negatively charged coating 215 comprising graphene oxide plus Bovine Serum Albumin (BSA). The combination of GO and BSA creates a surface that inherently maintains a negative charge. GO's functional groups can ionize to provide negative charges, while BSA stabilizes these charges through its protein structure. As blood flows through the aorta, the mechanical interaction between the blood and the device 200, or the struts 210 and/or 212 of the device 200 induces a triboelectric effect. The GO-BSA coating ensures that the triboelectric effect continuously generates and maintains the negative charge necessary to repel negatively charged clots, and microparticles. Some alternative microparticles that can be integrated with the negatively charged coating for enhanced electronegative surface charges and biocompatibility may include gold microparticles, silver microparticles, alumina (Al₂O₃) microparticles, and/or titanium dioxide (TiO₂) microparticles.

In addition, and in the exemplary embodiment shown in FIG. 2E, device 200 may further comprise one or more radiopaque markers 214 located proximally and/or distally on device 200 to aid the placement of device 200 within a body. For example, in at least one embodiment, one or more radiopaque markers 214 are positioned on the filter portion 204 in a location(s) relative to the convex struts 210 of the device 200. Accordingly, when the device 200 is positioned within an artery, the one or more markers 214 on the device 200 can be visualized to identify the orientation of the convex struts 210 relative to the direction of the blood flow.

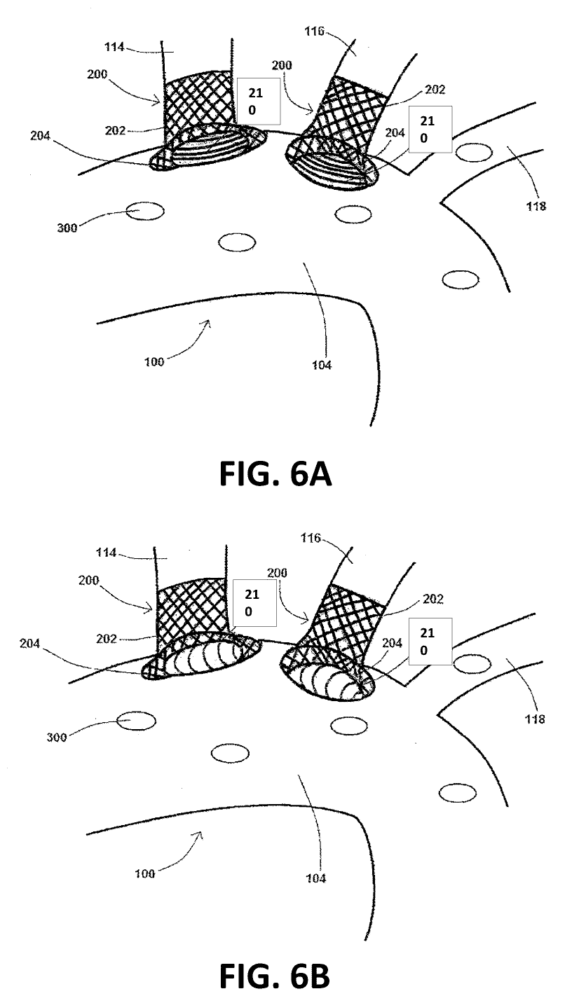

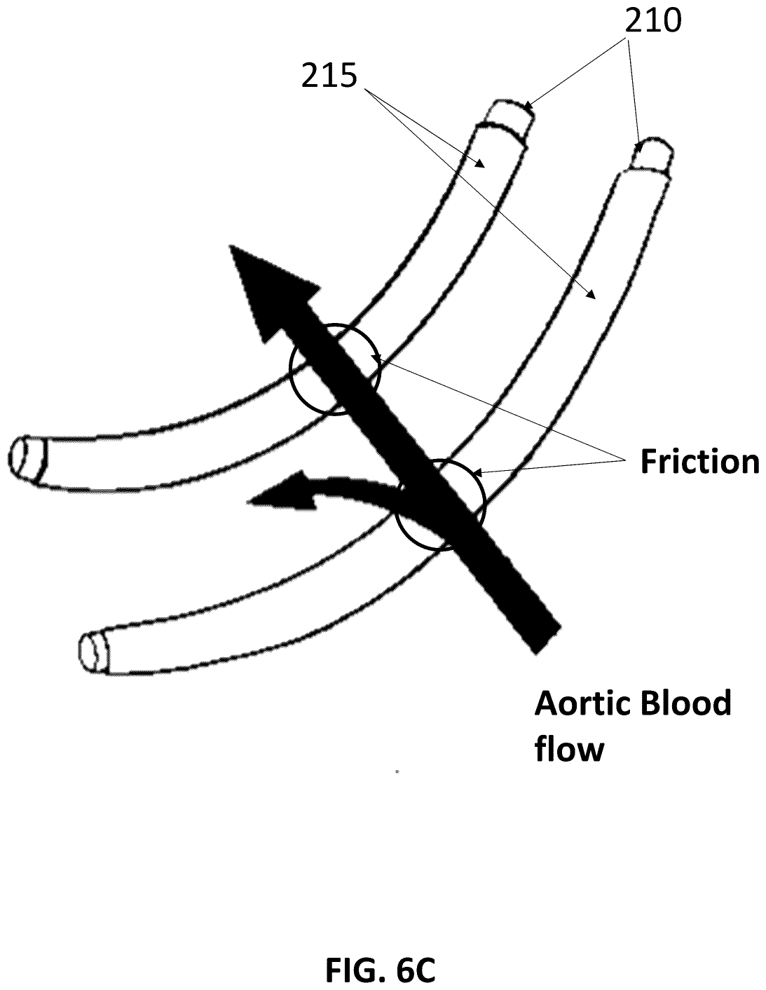

Exemplary devices for the prevention of stroke positioned within a portion of an aorta are shown in FIGS. 6A and 6B. While the devices 200 illustrated in FIGS. 6A and 6B both comprise a filter portion 204 and a stent portion 202 having extension mesh 206, it will be understood that any embodiments of the device 200 of the present disclosure can be positioned pursuant to and are capable of the same functionality described in connection with FIGS. 6A and 6B.

As shown in FIGS. 6A and 6B, two devices 200 are positioned within arteries branching from aorta 100, with one device 200 positioned partially within innominate artery 114 and another device 200 positioned partially within left common carotid artery 116. While two devices 200 are illustrated in FIGS. 6A and 6B positioned partially within innominate artery 114 and left common carotid artery 116, it will be understood that the devices 200 can be positioned in any of innominate artery 114, left common carotid artery 116 and/or left subclavian artery 118. It will also be understood that a third device 200 of the present disclosure can be included and positioned in the left subclavian artery pursuant to and is capable of the same functionality described in connection with FIGS. 6A and 6B. Device 200 within innominate artery 114 is positioned such that stent portion 202 is positioned within a portion of innominate artery 114 extending from aortic arch 104 and filter portion 204 prevents device 200 from advancing further into innominate artery 114. Similarly, a device 200 is shown in FIGS. 6A and 6B positioned within left common carotid artery 116 such that stent portion 202 is located within a portion of left common carotid artery 116 extending from aortic arch 104 and filter portion 204 prevents device 200 from advancing further into left common carotid artery 116. In at least one embodiment, filter portion 204 completely covers and exceeds the size of the entrance of the artery in which device 200 is positioned. In an exemplary embodiment of device 200 positioned within an artery as referenced herein, the distal cylindrical portion of the stent (stent portion 202 of device 200) additionally or alternatively anchors device 200 by applying radial force to the arterial walls of the artery in which device 200 is placed. In this manner, both the stent portion 202 and the filter portion 204 may act to anchor the device 200 in place when positioned within an artery.

As shown in the exemplary embodiments of device 200 shown in FIG. 6A, convex struts 210 are aligned in a direction similar to the flow of blood within aorta 100. In such an alignment, and as blood flows through aorta 100, an embolus 300 present within aorta 100 (specifically within the aortic arch 104) would be guided by the blood flow along convex struts 210 and across the proximal opening of the aortic branch. As shown in FIG. 6B, the convex struts 210 of both devices 200 are aligned in a direction approximately perpendicular to the flow of blood within aorta 100. In such an alignment, an embolus 300 present within aorta 100 would contact convex struts 210 and be deflected therefrom with little or no risk of embolus 300 being trapped therein. As referenced herein, convex struts 210 may also be positioned in the direction of (i.e., approximately parallel with), or in an oblique manner relative to, blood flow within the aortic arch 104 as shown in FIG. 6A. In application, the device 200 may be positioned within an artery to achieve any orientation of the convex struts 210 relative to the flow field that may be desired in accordance with patient specifications and/or user preference.

Positioning the devices 200 as shown in FIGS. 6A and 6B prevents an embolus 300 from entering the innominate artery 114 and the left common carotid artery 116 but allows the embolus 300 to enter the left subclavian artery 118. Because the innominate and left common carotid arties 114, 116 supply blood flow to the brain, in this example, the devices 200 thus prohibit the embolus 300 from advancing to the brain vascular system, thereby significantly reducing a patient's risk of ischemic stroke. Instead, the embolus 300 is allowed to flow into other arteries—such as the femoral or iliac arteries, for example—where such embolus 300 can be filtered from or sucked out of the blood stream using an appropriate medical procedure. In other words, such an arrangement of devices 200 may effectively prevent a patient from having a stroke by deflecting any embolus 300 present in the blood stream away from the vessels that feed the brain and instead routing such emboli 300 to a location where they may be easily and safely removed.

In summary, and as described above with respect to FIGS. 6A and 6B, for example, the present disclosure provides a device 200, which may be referred to as a percutaneous carotid emboli rerouting device, configured for individual delivery to an artery given off by the aortic arch 104 (namely the innominate artery 114, the left common carotid artery 116, and the left subclavian artery 118) to avoid the passage of embolic or thromboembolic material (an embolus 300, which may be, for example, a clot, calcium, etc.) to the brain vascular system. Furthermore, the present disclosure provides for the provision of more than one of these devices 200 to the arteries off the aortic arch 104 such that an arrangement of devices 200 prevents thromboembolic stroke in patients with different cardiovascular diseases from cardiac origin.

At least one goal of the devices, systems, and methods of the present disclosure is to reroute an embolus distally to the arterial system (iliac or femoral arteries) to avoid disabling stroke, decrease mortality and avoid physical impairment with a poor quality of life. As previously mentioned, unlike stroke, medical or surgical treatment of the peripheral arterial embolus (fibrinolitic drugs, surgical embolectomy, or endovascular embolus suction) can be provided with little residual effect. This may be particularly useful to patients who have undergone medical procedures associated with a high risk of stroke and/or blood clots being released following the procedures (e.g., transcatheter aortic valve implantation (“TAVI”), mitral valve replacement, calcific mitral valve insufficiency, balloon dilation, etc.). For example, the general risk of stroke after TAVI is about three percent (3%), which increases to about six to ten percent (6-10%) thirty days following the procedure, and again to about seventeen to twenty-four percent (17-24%) one year following the procedure. As such, while TAVI (or similar procedures) is often used to repair a patient's heart and/or circulatory system, the procedure often results in brain damage due to its side-effect of increasing the occurrence of blood clots.

The devices, systems and methods of the present disclosure can be used in connection with such patients to divert the resulting clots. Moreover, the devices, systems and methods described herein are also particularly applicable to patients who cannot receive anticoagulants, are prone to clots forming in the left atrial appendage and entering the bloodstream, or simply present an elevated risk for brain damage due to stroke. The risk of brain damage can also generally be reduced with the elderly by employing the devices, systems and methods disclosed herein.

An exemplary embodiment of a system for preventing stroke of the present disclosure is shown in FIG. 7. As shown in FIG. 7, system 400 comprises a hypotube 402 having a distal end and a proximal end, and in at least one exemplary embodiment, hypotube 402 comprises a folder 404 coupled to the distal end of hypotube 402. In the embodiment shown in FIG. 7, system 400 further comprises a device 200, whereby a stent portion 202 of device 200 is shown positioned within at least part of folder 404 and a filter portion 204 of device 200 is positioned within at least part of a sleeve 406 and around hypotube 402 proximally of folder 404. Sleeve 406, as shown in this exemplary embodiment, slidingly engages hypotube 402 and may be moved in a forward or backward direction as indicated by the arrow in the figure.

In at least one embodiment, device 200 is an autoexpandable metallic stent mounted over a hypotube 402 as shown in FIG. 7. Device 200 may be compressed by sleeve 406 and folder 404 such that both the stent portion 202 and the filter portion 204 are in their collapsed positions. In at least one embodiment, at least part of system 400 has a diameter of 7 Fr to 8 Fr (2.3 to 2.7 mm), with an exemplary device 200 having a compressed diameter of about 1.8 to 2.0 mm.

FIGS. 8A and 8B show exemplary embodiments of at least portions of systems for preventing stroke of the present disclosure. As shown in FIG. 8A, an exemplary system 400 comprises hypotube 402 to which folder 404 is coupled thereto. System 400, as shown in FIGS. 8A and 8B, further comprises sleeve 406 slidingly engaged around hypotube 402. Device 200 may be positioned at least partially within folder 404 and sleeve 406 prior to deployment, whereby the stent portion 202 of device 200 may be positioned within at least part of folder 404 in a collapsed position, and whereby the proximal portion (i.e., the filter portion 204) of device 200 may be positioned within at least part of a sleeve 406 in a collapsed position (as shown in FIG. 7).

As shown in FIG. 8A, device 200 may be partially deployed as follows. First, and in an exemplary method of positioning a stent within a body, a wire 500 (a guide wire, for example) may be advanced within a body at or near a desired location of device 200 deployment. When wire 500 has been advanced, hypotube 402, along with any portions of system 400 coupled to hypotube 402, may be advanced along wire 500 within the body. As shown in FIGS. 8A and 8B, initial advancement of at least a portion of system 400 may comprise advancement of hypotube 402, folder 404, sleeve 406, and device 200 positioned within folder 404 and sleeve 406.

When device 200 has been positioned within a body at or near a desired position, sleeve 406 may be withdrawn toward the proximal end of hypotube 402 (in the direction of the arrow shown in the figure). This step may be performed prior to, during, or after the step of positioning the distal end of hypotube 402 within a vessel (for example, a vessel branching off the aortic arch 104). As sleeve 406 is slid toward the proximal end of hypotube 402, the filter portion 204 of device 200 is allowed to expand as shown in FIG. 8A. While at this step the filter portion 204 is deployed, the stent portion 202 remains within the folder 404. Accordingly, the stent portion 202 remains undeployed and does not yet engage or anchor to an arterial wall.

Further deployment of device 200 within a body is shown in FIG. 8B. As shown in FIG. 8B, and upon movement of folder 404 away from device 200 (in a direction shown by the arrow in the figure, for example), stent portion 202 of device 200 may deploy as shown in FIG. 8B. As folder 404 is moved away from device 200 (by, for example, advancement of hypotube 402 within a body), stent portion 202 of device 200 is no longer positioned within folder 404, thereby permitting expansion/deployment of stent portion 202.

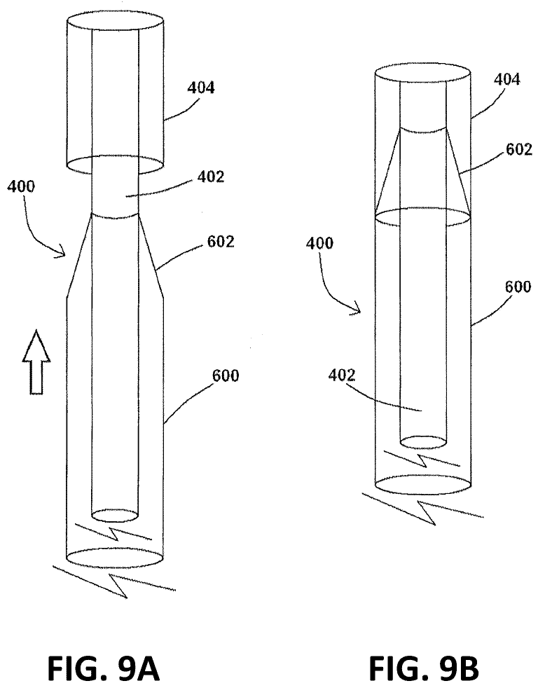

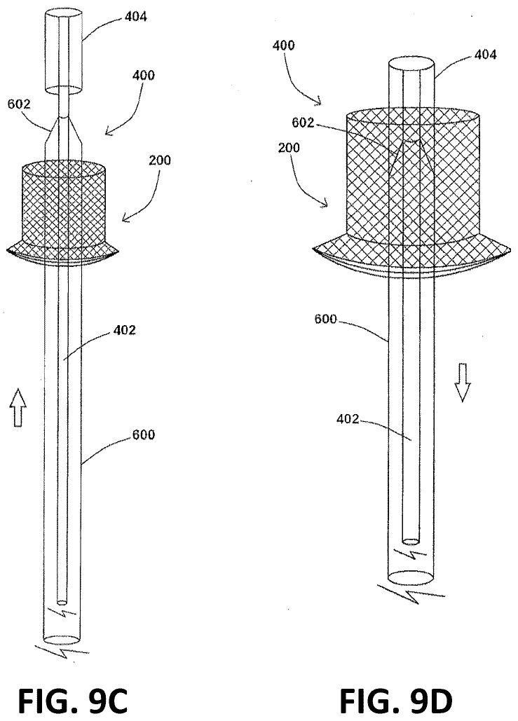

FIGS. 9A and 9B show exemplary embodiments of at least a portion of a system for preventing stroke. In at least one embodiment, system 400 comprises a conical dilator 600 slidingly engaged around a hypotube 402 coupled to a folder 404. As shown in FIG. 9A, an exemplary conical dilator 600 may comprise a tapered distal end 602, wherein the tapered distal end 602 is sized and shaped to engage the inside of folder 404. To engage folder 404, conical dilator 600 may slide along hypotube 402 in a direction indicated by the arrow in FIG. 9A. An exemplary embodiment of the engagement of conical dilator 600 and folder 404 is shown in FIG. 9B.

Engagement of conical dilator 600 with folder 404, as shown in FIGS. 9A and 9B, may facilitate the removal of at least a portion of system 400 from a body after positioning device 200. For example, and as shown in FIGS. 8A and 8B, after deployment of device 200 within a body, the portion of system 400 comprising folder 404 is positioned, for example, further within a vessel than device 200. Removal of the portion of the system 400 comprising hypotube 402 and folder 404 would require, for example, pulling that portion of system 400 back through device 200. As shown in the exemplary embodiments of FIGS. 8A-9B, folder 404 may, for example, become caught on device 200 and/or a portion of a body, preventing effective removal of that portion of system 400.

In at least one embodiment, and by engaging folder 404 with conical dilator 600, folder 404, along with the portion of system 400 coupled to folder 404, may be removed from a body after placement of a device 200 as shown in FIGS. 9C and 9D. As shown in FIG. 9C, and after a device 200 has been deployed, a user of system 400 may slide a conical dilator 600 along hypotube 402 in a direction indicated by the arrow. Conical dilator 600, in the example shown in FIGS. 9C and 9D, is sized and shaped to fit within the spaces between convex struts 210 of device 200. After conical dilator 600 has engaged folder 404, as shown in FIG. 9D, when hypotube 402 is withdrawn from the body in a direction indicated by the arrow, folder 404 is also removed from the body without becoming caught on device 200.

In at least one embodiment of a system for preventing stroke of the present disclosure, system 400 comprises a device 200, a hypotube 402, and a folder 404 coupled to hypotube 402 at or near the distal end of hypotube 402. Device 200, in at least one embodiment, may be autoexpandable, i.e. device 200 has a “memory” allowing it to expand to a native configuration after being retracted/compressed to fit within, for example, folder 404 and sleeve 406. System 400, in at least one embodiment, may further comprise, or be used in connection with, a femoral catheterization kit known and used in the marketplace.

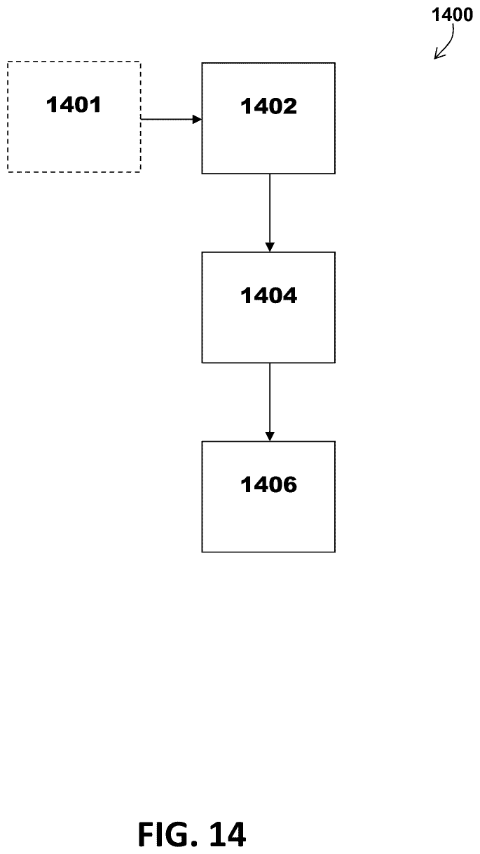

Now referring to FIG. 14, at least one method of preventing stroke will now be described using the components of the previously described systems for reference and explanatory purposes. Primarily, at step 1402, the device 200 is positioned within a body. In at least one exemplary embodiment of the present disclosure, the percutaneous placement of the percutaneous carotid emboli rerouting device (device 200) may be performed in an angiography procedure room. Prior to positioning device 200 at step 1402, a user may optionally perform a contrast aortogram, for example, to map out the aortic arch 104 and where the cerebral vessels merge with aortic arch 104 (optional step 1401). For safety, patient preparation and sterile precautions are recommended as for any angioplasty procedure.

In at least one embodiment of the method 1400 for preventing stroke, the optional step 1401 of the method 1400 additionally or alternatively comprises performing a percutaneous angiogram using technique(s) known in the art under local anesthesia. As referenced above, the percutaneous angiogram maps the aortic arch 104 so that a user of a device 200 and/or system 400 of the present disclosure can, for example, select an appropriately-sized device 200 and/or system 400 (or portion(s) thereof) when performing the procedure.

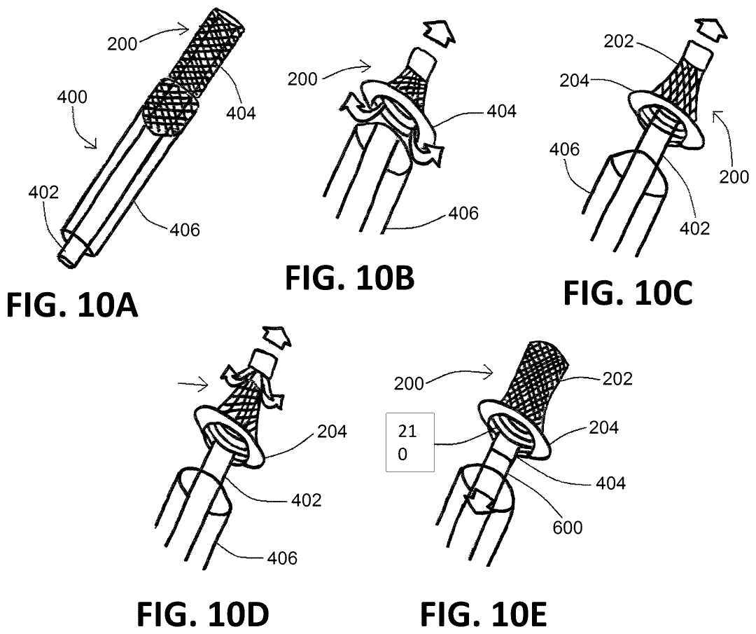

At step 1402, to facilitate positioning the device 200 within a body, a user may introduce a wire 500 (such as guide wire as shown in FIG. 8A) to reach the innominate artery 114, the left common carotid artery 116 and/or the left subclavian artery 118. After wire 500 has been positioned, portions of system 400 may be mounted over the guide wire 500 and progressed to the level of the entrance of the innominate artery 114, the left common carotid artery 116 and/or the left subclavian artery 118. Said portions of system 400 may include hypotube 402 and a folder 404 distally mounted thereto, and may further comprise a sleeve 406, wherein an exemplary device 200 may be positioned at least partially within folder 404 and sleeve 406, as shown in FIG. 10A. After the device(s) 200 are properly positioned at step 1402, the method 1400 advances to step 1404 where the device(s) 200 are deployed.

Deployment of device 200 at step 1404, in an exemplary embodiment of a method of the present application for performing the same, is as a follows. Under fluoroscopy, sleeve 406 may be pulled back to allow the delivery of the proximal portion of the stent (the filter portion 204 of device 200) as shown in FIG. 10B. The diameter of filter portion 204 that exceeds the diameter of the innominate artery 114, the left common carotid artery 116 and/or the left subclavian artery 118 impedes the progression of device 200 within said arteries, thus giving the user/operator time to deliver and anchor the second portion of the stent (the stent portion 202 of device 200) by, for example, forward progression of hypotube 402 as shown in FIGS. 8B and 10C. In addition to preventing the device 200 from progressing within the artery, when the filter portion 204 is expanded upon delivery to the artery of interest, such structures also provide support over the aortic wall of the aortic arch 104 at the level of proximal aortic ostium in which the device 200 is deployed.

In at least one embodiment, deployment of the device 200 at step 1404 may be facilitated through the use of radiopaque markers 214. Where the device 200 comprises radiopaque markers 214, prior to anchoring the stent portion 202 of the device 200, such markers 214 can be used to assist with ensuring proper alignment. Specifically, the user/operator can visualize the radiopaque markers 214 through fluoroscopy or other technology and rotate the device 200 accordingly so that the convex struts 210 are positioned as desired relative to the direction of blood flow within the aortic arch 104. In this manner, the radiopaque markers 214 can facilitate placement and orientation of the device 200. In various embodiments, device 200 can be positioned approximately perpendicular to, or in a direction of (i.e. approximately parallel with), or in an oblique manner relative to, blood flow in the aortic arch 104, and can even be positioned/deployed in an oblique manner (not parallel or perpendicular), should such a deployment be desired.

When device 200 has been positioned at step 1402 and deployed at step 1404, the method 1400 may advance to step 1406 where the hypotube 402 and folder 404 are removed from the body, for example, by introducing conical dilator 600 as described herein. In at least one example, the tapered distal end 602 of conical dilator 600 is advanced until it engages folder 404 of hypotube 402, as shown in FIGS. 9A-9D, 10D and 10E, effectively forming a single unit (conical dilator 600+hypotube 402+optionally wire 500 (not shown)). This “unit” may then be removed through the convex struts 210 as shown in FIG. 10E, and distally to the femoral artery for which at least part of system 400 was initially introduced.

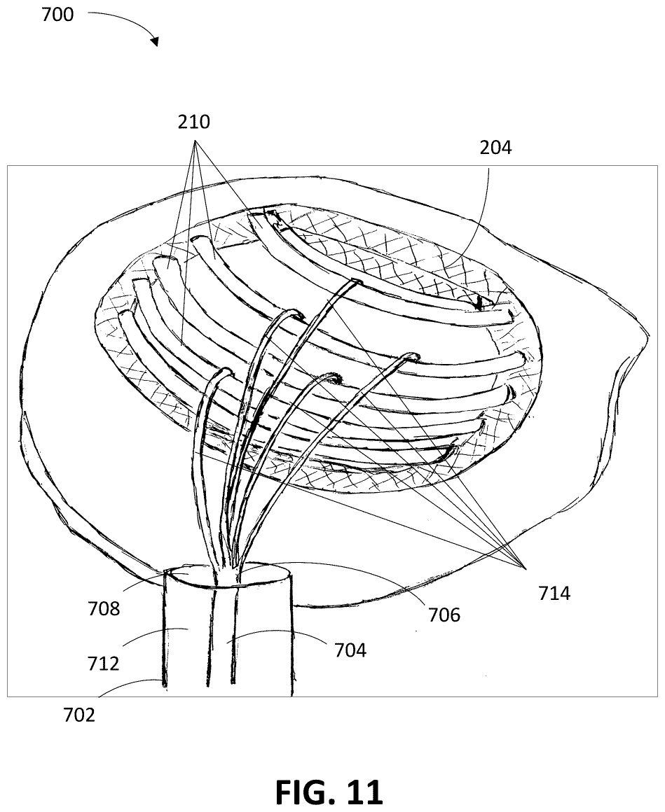

Now referring to FIGS. 11-13B, an exemplary system 700 for preventing stroke of the present disclosure is shown. At times, temporary placement of the filter portion 204 of the devices 200 disclosed herein may be desired (as opposed to chronic or permanent placement). In such cases, it is necessary to retrieve the filter portion 204 of the device 200, from the patient after a prescribed period of time has elapsed or other indications are observed. System 700 comprises a retrieval system for use in retrieving the filter portions 204 of device 200 previously positioned within an artery extending from the aortic arch 104.

System 700 comprises a sleeve catheter 702, a retrieval device 704, and at least one device 200. The sleeve catheter 702 is configured for intravascular insertion and advancement, and comprises an open distal end 708, a proximal end (not shown), and a lumen 712 extending therebetween. The retrieval device 704 is slidably disposed within the lumen 712 of the sleeve catheter 702 and comprises a proximal end (not shown) for manipulation by a user/operator and a distal end 706 configured for advancement through the open distal end 708 of the sleeve catheter 702. The distal end 706 of the retrieval device 704 further comprises one or more attachment portions 714 positioned thereon, each of which are configured to engage the filter portion 204 of device 200.

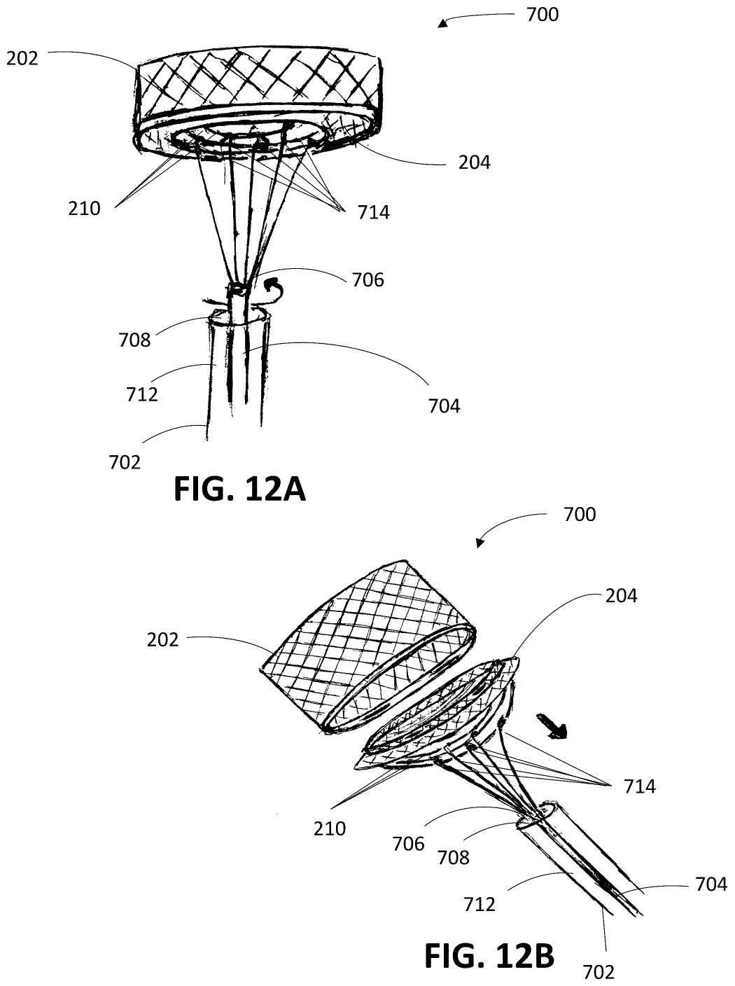

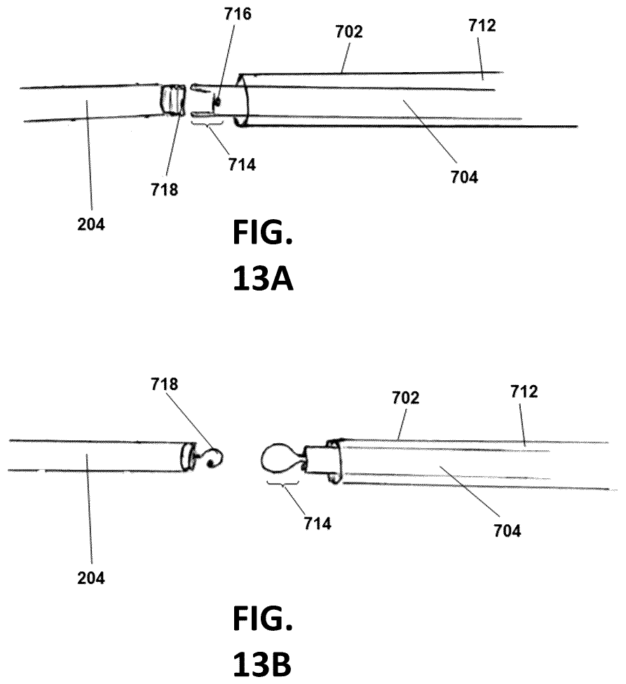

The retrieval device 704 may comprise any configuration suitable for slidably advancing through the lumen 712 and through the open distal end 708 of the sleeve catheter 702. For example, in the embodiments shown in FIGS. 11-12B, the retrieval device 704 comprises one or more wires. Alternatively, in the embodiment of FIGS. 13A and 13B, the retrieval device 704 comprises an elongated catheter having one or more attachment portions 714 configured to engage the filter portion 204 of the device 200. Furthermore, the proximal portion (the filter portion 204) of the device 200 may be additionally configured to engage or receive the attachment portion(s) 714 of the retrieval device 704.

Now referring back to FIGS. 11-12B, an embodiment of a system 700 for retrieving the filter portion 204 of device 200 is shown. This embodiment of the system 700 has a retrieval device 704 comprising one or more wires slidably disposed within the lumen 712 of the sleeve catheter 702. Each of the wires of the retrieval device 704 of this embodiment comprises an attachment portion 714 configured to securely grab the convex struts 210 of filter portion 204. For example, an attachment portion 714 may be curved or comprise a hook capable of grabbing one of the convex struts 210 of the filter portion 204. Alternatively, the attachment portion 714 may comprise any other configuration capable of securely grabbing at least one of the convex struts 210 of the filter portion 204 such that a sufficient amount of force can be exerted on the filter portion 204.

While FIGS. 11A-12 illustrate embodiments of the system 700 comprising a retrieval device 704 having wires, the retrieval device 704 of the system 700 may comprise any configuration suitable for slidably advancing through the lumen 712 and the open, distal end 708 of the sleeve catheter 702. FIGS. 13A and 13B show two non-limiting examples of such alternative embodiments of a retrieval device 704. In these embodiments, the retrieval device 704 comprises an elongated catheter having an attachment portion 714 on or near its distal-most end. Likewise, the proximal portion (filter portion 204) of the device 200 may be configured to correspond with the attachment portion 714 of the retrieval device 704. For example, in the embodiment shown in FIG. 13A, the attachment portion 714 of the retrieval device 704 defines a cavity having female threads disposed therein and a magnet 716, while the filter portion 204 of the device 200 comprises a corresponding portion 718 having male screw threads and a magnet. Similarly, FIG. 13B shows an embodiment where the attachment portion 714 of the retrieval device 704 comprises a lace and the corresponding portion 718 of the filter portion 204 comprises a corresponding hook tip. Accordingly, in each of the aforementioned embodiments, the device 200 may be easily engaged by the attachment portion 714 of the retrieval device 704.

After the attachment portion 714 of the retrieval device 704 is securely coupled with the filter portion 204 of device 200 (via the corresponding portion 718 or otherwise), a user/operator can manipulate the proximal end (not shown) of the retrieval device 704 and thus manipulate the filter portion 204. In this manner, a user/operation may rotate the filter portion 204 relative to the stent portion 202 and thus disengage the filter portion 204 from the stent portion 202 as described above with regard to the device 200 in FIGS. 4A-5D. After the filter portion 204 is disengaged, it is caused to shift from the expanded position to the collapsed position by pulling the retrieval device 704 in a retrograde manner into the sleeve catheter 702 thereby forcing the filter portion 204 into its collapsed position as it is pulled with the retrieval device 704 into the sleeve catheter 702, and retrieval device 704 (and thus the collapsed filter portion 204) is then slidably removed from the sleeve catheter 702 and the patient's body.

The various devices, systems, and methods for preventing stroke of the present disclosure have various benefits to patients with various diseases and/or disorders of the heart and/or circulatory system. For example, patients with chronic atrial fibrillation (non-valvular atrial fibrillation), recurrence transient ischemic attack, atrial fibrillation and anticoagulation contraindications, and/or left atrial appendage thrombosis may have their risk of stroke either reduced or eliminated by way of an exemplary devices, systems, and/or method of the present disclosure. In addition, patients with acute myocardial infarct with left ventricular thrombus, atrial flutter or fibrillation (ablation and pulmonary vein isolation), cardiomyopathy with left ventricular enlargement, non-obstructive thrombus of a mechanical heart valve, patent foramen ovale (cryptogenic ischemic stroke) and/or an acute infection endocarditis with valve vegetation without valve insufficiency under medical treatment (vegetation >1 cm which currently oblige to surgical remotion) may also benefit from the present disclosure.

Furthermore, it is noted that the various devices, systems, and methods for preventing stroke of the present disclosure have advantages as compared to anticoagulant and antiplatelet therapies, as not all patients are suitable for such therapies (given the high risk of bleeding, for example), and the relative cost of such therapies, which would be substantially higher as compared to the devices and systems as referenced herein. The various devices and systems would be useful for various aortic arch configurations, noting that there is diversity among arches.

While various embodiments of devices, systems, and methods for the prevention of stroke have been described in considerable detail herein, the embodiments are merely offered by way of non-limiting examples of the disclosure described herein. It will therefore be understood that various changes and modifications may be made, and equivalents may be substituted for elements thereof, without departing from the scope of the disclosure. Indeed, this disclosure is not intended to be exhaustive or to limit the scope of the disclosure.

Further, in describing representative embodiments, the disclosure may have presented a method and/or process as a particular sequence of steps. However, to the extent that the method or process does not rely on the particular order of steps set forth herein, the method or process should not be limited to the particular sequence of steps described. Other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

1. A device for the prevention of stroke, the device comprising:
a stent portion having a first end and a second end and sized and shaped to fit within an artery extending from an aortic arch; and
a filter portion positioned at the second end of the stent portion and sized and shaped to prevent the device from advancing into the artery extending from the aortic arch in which the stent portion may be positioned, the filter portion comprising at least two sets of two or more parallel, convex struts positioned across an opening defined within the second end of the stent portion, the at least two sets of two or more parallel, convex struts configured to divert an embolus from entering the artery when the first end of the stent portion is positioned within the artery.

2. The device of claim 1, wherein the at least two sets of two or more parallel convex struts comprises a first set of parallel convex struts and a second set of parallel convex struts.

3. The device of claim 2, wherein when the device is positioned within the artery extending from an aortic arch, the first set of two or more parallel convex struts are positioned approximately perpendicular to a direction of blood flow within the aortic arch and the second set of two or more parallel convex struts are positioned perpendicular to the first set of parallel convex struts.

4. The device of claim 2, wherein when the device is positioned within the artery extending from an aortic arch, the first set of two or more parallel convex struts are positioned in a direction of blood flow within the aortic arch and the second set of two or more parallel convex struts are positioned perpendicular to the first set of parallel convex struts.

5. The device of claim 1, wherein the filter portion is removably attached to the second end of the stent portion.

6. The device of claim 1, wherein the stent portion has a length between about 0.8 cm to about 2.5 cm.

7. The device of claim 1, wherein the stent portion has a diameter between about 6 mm to about 8 mm when the extension portion is in an expanded configuration.

8. The device of claim 1, wherein the stent portion has a diameter between about 1.8 mm to about 2.0 mm when the extension portion is in a compressed configuration.

9. The device of claim 1, wherein the device is comprised of a material selected from the group consisting of stainless steel, cobalt-chromium-nickel-molybdenum-iron alloy, tantalum, nitinol, nickel-titanium, polymer materials, and a shape-memory polymer.

10. The device of claim 1, wherein the filter portion comprises a planar flange.

11. The device of claim 1, further comprising:
one or more radiopaque markers positioned upon the filter portion.

12. The device of claim 11, wherein the one or more radiopaque markers are positioned relative to the first set of two or more parallel convex struts.

13. The device of claim 11, wherein when the device is positioned within the artery extending from an aortic arch, the one or more radiopaque markers facilitate alignment of the device so that the first set of two or more parallel convex struts are positioned approximately perpendicular to a direction of blood flow within the aortic arch.

14. The device of claim 11, wherein when the device positioned within the artery extending from an aortic arch, the one or more radiopaque markers facilitate alignment of the device so that the first set of two or more parallel convex struts are positioned in a direction of blood flow within the aortic arch.

15. The device of claim 1, wherein the diameter of each strut of the at least two sets of two or more parallel convex struts is between about 0.25 mm and 0.5 mm.

16. The device of claim 2, wherein each strut of the first set of two or more parallel convex struts are positioned between about 0.5 mm to 1.5 mm from one another, and each strut of the second set of two or more parallel convex struts are positioned between about 0.5 mm to 1.5 mm from one another.

17. The device of claim 1, wherein the at least two sets of two or more parallel, convex struts are negatively charged.

18. The device of claim 1, wherein the at least two sets of two or more parallel, convex struts are coated with a coating comprising graphene oxide.

19. The device of claim 18, wherein the coating further comprises bovine serum albumin.

20. The device of claim 18, wherein the coating further comprises at least one microparticle from the group consisting of gold microparticles, silver microparticles, alumina microparticles, and titanium dioxide microparticles.

21. A system for preventing stroke, the system comprising:
a device for the prevention of stroke, the device comprising:
a stent portion having a first end and a second end and sized and shaped to fit within an artery extending from an aortic arch; and
a filter portion positioned at the second end of the stent portion and sized and shaped to prevent the device from advancing into the artery extending from the aortic arch in which the stent portion may be positioned, the filter portion comprising at least two sets of two or more parallel, convex struts positioned across an opening defined within the second end of the stent portion, the at least two sets of two or more parallel, convex struts configured to divert an embolus from entering the artery when the first end of the stent portion is positioned within the artery;
a hypotube having a distal end and a proximal end;
a folder coupled to the distal end of the hypotube, the folder sized and shaped to receive at least a portion of the device for the prevention of stroke; and
a sleeve positioned circumferentially around the hypotube proximal to the folder, the sleeve sized and shaped to receive at least a portion of the device for the prevention of stroke.

22. The system of claim 21, wherein the at least two sets of two or more parallel convex struts of the device comprises a first set of parallel convex struts and a second set of parallel convex struts perpendicular to the first set of parallel convex struts.

23. The system of claim 21, wherein the filter portion of the device is removably attached to the second end of the stent portion of the device.

24. The system of claim 21, wherein the stent portion and the filter portion are autoexpandable.

25. The system of claim 21, wherein the device is comprised of a material selected from the group consisting of stainless steel, cobalt-chromium-nickel-molybdenum-iron alloy, tantalum, nitinol, nickel-titanium, polymer materials, and a shape-memory polymer.

26. The system of claim 21, wherein the filter portion of the device comprises a planar flange.

27. The system of claim 21, wherein the at least two sets of two or more parallel, convex struts are negatively charged.

28. The system of claim 21, wherein the at least two sets of two or more parallel, convex struts are coated with a coating comprising graphene oxide.

29. The system of claim 28, wherein the coating further comprises bovine serum albumin.

30. The device of claim 28, wherein the coating further comprises at least one microparticle from the group consisting of gold microparticles, silver microparticles, alumina microparticles, and titanium dioxide microparticles.
